(19) United States
(12) Patent Application Publication
(10) Pub. No.: US 2005/0042313 A1
(43) Pub. Date: Feb. 24, 2005

(54) Method to enhance the immune system and used for the prevention and treatment of infectious diseases

(76) Inventor: Virgilio Verzosa Ecarma, Paranaque (PH)

Correspondence Address: MOAZZAM LATIMER LLP, 1474 NORTH POINT VILLAGE CENTER #320, RESTON, VA, 20194-1190, US

(21) Appl. No.: 10/956,097

(22) Filed: Oct. 4, 2004

Related U.S. Application Data
(63) Continuation of application No. 10/450,169, filed on Jun. 11, 2003.

(30) Foreign Application Priority Data
Dec. 19, 2000 (PH) I-2000-0003506

(51) Int. Cl. A61K 35/78; A61K 9/48

(52) U.S. Cl. 424/769; 424/452

(57) ABSTRACT

A pharmaceutical formulation for oral administration containing root, bark and wood of Philippine Narra tree (Pterocarpus Indicus) in dried powder form in sachet in a unit of 2 to 3 grams and made into a decoction of 8 ounces or in capsule in a unit of 100 to 300 milligrams at a dosage of three to six times a day can enhance the immune system of a person and can be used for the prevention and treatment of INFECTIOUS DISEASES.

### FIELD OF THE INVENTION

The present invention is directed to the decoction or as capsule of the root, bark and wood of the Philippine Narra tree (Pterocarpus Indicus) when taken three to six times a day to enhance the immune system and used for the prevention and treatment of INFECTIOUS DISEASES.

### BACKGROUND OF THE INVENTION

The applicant/inventor was awarded by the Philippine Bureau of Patents, Trademarks and Technology Transfer with Patents Nos. 27501, 29242, and 30978 for a pharmaceutical formulation for the oral administration containing bark and wood of the Philippine Narra tree (Pterocarpus Indicus) in dried powder form in a unit dosage from 1 to 5 grams which is effective for the treatment of diabetes mellitus, arthritis, hypertension and stone in the bladder. The Philippines Narra tree (Pterocarpus Indicus) has been used by the applicant/inventor as treatment for his Rheumatoid Arthritis. After years of use, it became apparent that the Philippine Narra can also enhance the immune system and the applicant/inventor has successfully treated immunosuppressed persons (HIV/AIDS patients) with it.

In this regard the applicant/inventor has applied with the Philippine Bureau of Patents, Trademarks and Technology Transfer for an Invention Patent entitled A METHOD USING THE PHILIPPINE NARRA TREE (PTEROCARPUS INDICUS, WILLD) AS HERBAL MEDICINE FOR THE ENHANCEMENT OF THE IMMUNE SYSTEM AND USED AS TREATMENT FOR HVI/AIDS, which is still pending.

The function of the immune system is to defend the body against invaders like viruses, fungi, cancer cells and other substances foreign to the body thus the body's immune system attacks and eliminates not only bacteria and other foreign substances but also cancer cells. The immune system's critical role in controlling cancer cell development is exemplified by an astounding statistic: Cancer is 100 times more likely to occur in people who take drugs that suppress the immune system (for example, because of an organ transplant or a rheumatic disease) than in people with normal immune systems. Moreover, it has been found that blood glucose is necessary for tumor and cancer cells to grow and proliferate. If blood glucose is considerably reduced; the cancer cells will wither and die. Philippine Narra has the property of lowering the blood glucose and also the property of enhancing the immune system of immunosuppressed persons (HIV/AIDS) which make it ideal to effectively treat cancer. In this regard the applicant/inventor has also applied with the Philippine Bureau of Patents, Trademarks and Technology Transfer for an Invention Patent entitled THE PHILIPPINE NARRA TREE (Pterocarpus Indicus, Willd.) AS HERBAL MEDICINE FOR THE TREATMENT OF CANCER, which is still pending. Although the immune system is intricate, its basic strategy is simple: to recognize the enemy, mobilize forces, attack and clean up the scene of the battle. Much of the immune system's machinery is geared toward killing or eliminating invading microbes once they have been recognized

### SUMMARY OF THE INVENTION

The invention relates to the discovery of the medicinal value of the root, bark and wood of the Philippine Narra tree (Pterocarpus Indicus) in decoction or powder form to enhance the immune. The advantages of this are its having no side effects, affordable and easy to manufacture. Other advantageous features of this invention will become apparent from the reading of the following detailed description.

### DETAILED DESCRIPTION OF THE INVENTION

The newly cut root, bark, and wood of the Philippine Narra tree (Pterocarpus Indicus) are scrubbed of any dirt and extraneous materials. It is washed twice with clean water and again washed with chlorinated water of 20 PPM of Chlorine. It is air-dried for several weeks. It is grounded to a powder form and dried in an oven to a moisture content of less than 10%.
One process is to pack the powder form in capsules at 100 to 300 milligrams each.
Another process is to pack the powder form in sachet at 2 to 3 grams each.
Another process is to boil for at least 15 minutes the powder form at 2 to 3 grams per 8 ounces of water and store the decoction in sterile bottle and refrigerate.

A Community Based Health Program (an NGO) invited the applicant/inventor to share his above-mentioned formulation for free to the less fortunate inhabitants of Barangay Tala, Kaloocan City. This is for the various illnesses that the formulation is suited for. The Jose N. Rodriguez Memorial Hospital that primarily treats leprosy patients is also located at Barangay Tala, Kaloocan City. Because the applicant/inventor patented invention shows that it can boost the immune system, many diseases were alleviated such as colds, flu, skin infections, acne, urinary track infections, respiratory infections, cysts, and many other infectious diseases including leprosy.

1. A pharmaceutical formulation from the root, bark and wood of the Philippine Narra tree (Pterocarpus Indicus) in dried powder form in sach of 2 to 3 grams which is made into a decoction and administered in an effective dosage to enhance the immune system and can be used as prevention and treatment of infectious diseases.

2. A pharmaceutical formulation, which is the root, bark and wood of the Philippine Narra tree (Pterocarpus Indicus) in dried powder form in a capsule of 100 to 300 milligrams and administered in an effective dosage to enhance the immune system and can be used as prevention and treatment of infectious diseases.
